(19) United States
(12) Patent Application Publication
(10) Pub. No.: US 2005/0175693 A1
(43) Pub. Date: Aug. 11, 2005

(54) Substained release formulation of protein and preparation method thereof

(75) Inventors: Hee-Yong Lee, Daejeon (KR); Jung-Soo Kim, Daejeon (KR); Ji-Suk Lee, Daejeon (KR); Jung-In Kim, Daejeon (KR); Yun-Mi Seo, Chungcheongnam (KR); Chae-Jin Lim, Daejeon (KR); Sung-Kyu Kim, Daejeon (KR); Young-Hwan Jung, Daejeon (KR); Seung-Gu Chang, Daejeon (KR); Ho-Il Choi, Daejeon (KR)

Correspondence Address: GREENBLUM & BERNSTEIN, P.L.C., 1950 ROLAND CLARKE PLACE, RESTON, VA, 20191, US

(73) Assignee: PEPTRON CO., LTD, DAEJEON (KR)

(21) Appl. No.: 10/512,090

(22) PCT Filed: May 9, 2003
(86) PCT No.: PCT/KR03/00921

(30) Foreign Application Priority Data
May 9, 2002 (KR) 10-2002-0025522

(51) Int. Cl. A61K 38/17; A61K 31/737; A61K 9/22

(52) U.S. Cl. 424/468; 514/2; 514/54

(57) ABSTRACT

The present invention relates to a sustained release formulation comprising protein as an active ingredient, and a preparation method thereof. According to the present invention, the sustained release formulation contains protein drugs that are encapsulated in biodegradable hydrophobic matrices as pharmaceutically active forms by forming complexes with sulfated polysaccharides. The sustained release formulation prepared by the present invention can be used to effectively treat a disease without frequent injections by keeping the protein concentration at a sufficiently high level for a long period when injected in vivo once.

### FIELD OF THE INVENTION

The present invention relates to a sustained release formulation of protein from which the drug is continuously released in vivo as a therapeutically active form in a controlled manner, and a preparation method thereof.

### BACKGROUND ART

Most protein drugs have poor oral absorption and very short half-lives after being administered by parental routes such as intravenous, subcutaneous and intramuscular injections. As a result, repetitive injection, infusion or sustained release dosage forms are required to obtain a desired therapeutic efficacy in a patient. For the purpose of obtaining in vivo sustained release of therapeutic proteins and peptides for a prolonged period, biodegradable natural and synthetic polymeric materials have been extensively studied for the carriers [Heller, J. et al., Biomaterials, 4, 262-266 (1983); Langer, R., Science, 249, 1527-1533 (1990); Okada, H. and Toguchi, H., Crit. Rev. Ther. Drug Carrier Syst., 12, 1-99 (1995)].

Among the biodegradable polymers, mostly aliphatic polyesters including polylactides (PLA), polyglycolides (PGA) and their copolymers (PLGA) have been investigated [DeLuca, P. P. et al., Biodegradable polyesters for drug and polypeptide delivery, in: El-Nokaly, M. A., Piatt, D. M., and Charpentier, B. A. (Eds.), Polymeric delivery systems, properties and applications, American Chemical Society, pp. 53-79 (1993); Park, T. G., Biomaterials, 16, 1123-1130 (1995); Anderson, J. M. and Shive, M. S., Adv. Drug. Del. Rev., 28, 5-24 (1997); Tracy, M. A. et al., Biomaterials, 20, 1057-1062 (1999)].

Natural materials being studied as matrices include lipids such as neutral lipids, fatty acids, waxes and their derivatives, proteins such as albumin, gelatin, collagen and fibrin, polysaccharides such as alginic acid, chitin, chitosan, dextran, hyaluronic acid, and starch. Due to the hydrophilic nature of matrix, it is very difficult to attain a release duration of several days or weeks when using proteins and polysaccharides as matrices. Alternatively, the release duration can be sustained for several days, weeks, and even months when synthetic polyesters and natural lipids are used as matrices.

In the present case, we mainly focused on synthetic polyesters and lipids as matrix materials. Several methods including solvent extraction and evaporation, phase separation and spray drying can be used to encapsulate protein drugs into hydrophobic matrices [McGee, J. P. et al., J. Controlled Rel., 34, 77-86 (1995); Gander, B. et al., J. Microencapsul., 12, 83-97 (1995); O'Donnell, P. B. and McGinity, J. W., Adv. Drug Del. Rel., 28, 25-42 (1997), U.S. Pat. No. 4,818,542, U.S. Pat. No. 5,942,253]. Due to the hydrophilic nature of most protein drugs, a water in oil in water (w/o/w) double emulsion solvent evaporation technique is frequently used for encapsulating protein into a biodegradable polymeric matrix. In the process, an aqueous protein solution is emulsified into a polymer-solvent phase, and this primary emulsion is further dispersed into a large volume of water phase containing an appropriate surfactant. Inevitably, protein drugs are exposed to a water/organic solvent interface. Most protein drugs are denatured and non-covalently aggregated during this primary emulsion stage. Consequently, the final product of protein-loaded microspheres typically show an initial burst release of native protein portions that are loosely bound to polymeric microspheres, followed by no significant release of irreversibly aggregated protein portions for any prolonged period [Kim, H. K. and Park, T. G., Biotechnol. Bioeng., 65, 659-667 (1999), Crotts, G. and Park, T. G., J. Microencapsul., 15, 699-713 (1998)].

Several efforts have been made to minimize denaturation and aggregation of protein during the encapsulation process.

Some stabilizing effects had been obtained through the use of excipients such as trehalose, mannitol, dextran, heparin and polyethylene glycol in the aqueous protein solution [U.S. Pat. No. 5,804,557, Cleland, J. L. and Jones, A. J. S., Pharm. Res., 13, 1464-1475 (1996), Cleland, J. L. et al., Pharm. Res., 14, 420-425 (1997), Pean, J. M. et al., Pharm. Res., 16, 1294-1299 (1999), Sanchez, A. et al., Int. J. Pharm., 185, 255-266 (1999), Lavelle, E. C. et al., Vaccine, 17, 516-529 (1999)]. These excipients seemed to partly prevent protein denaturation by forming a hydration layer around the protein and reducing the protein-organic solvent interactions. Use of a solid protein powder instead of an aqueous protein solution was another effort to minimize exposure of protein to a water-organic solvent interface [Cleland, J. L. and Jones, A. J. S., Stable formulations of recombinant hGH and interferon- [symbol] for microencapsulation in biodegradable microspheres, 13, 1464-1475 (1996); Iwata, M. et al., J. Microencapsul., 16, 49-58 (1999)].

This process was successfully applied to making poly(lactide-co-glycolide) microspheres containing the human growth hormone (hGH), and the product (Lutropin Depot™) was approved by the US FDA. In said process, Zn²⁺-stabilized hGH powders are suspended in a PLGA-methylene chloride solution, and this solution is atomized into liquid nitrogen layered onto frozen ethanol. As the temperature increases, melted ethanol extracts methylene chloride from the frozen droplet, and solidification of the microspheres can occur [U.S. Pat. No. 5,019,400, U.S. Pat. No. 5,654,010].

However, it has recently been reported that this hGH depot formulation has a relatively lower bioavailability (33-55%) than the earlier daily injection formulation of hGH [Cleland, J. L. et al., Emerging protein delivery methods, Current Opinion in Biotechnology, 12, 212-210 (2001)]. The low bioavailability of this depot formulation might be explained by high initial burst release and possible denaturation of unreleased proteins for a long time in vivo due to the slow degradation rate of PLGA.

Therefore, a method is needed for encapsulating the protein drug, in its fully active state, in biodegradable hydrophobic matrices while keeping the in vivo release rate of the drug in a controlled and sustained manner for several days and weeks without high initial release.

### SUMMARY OF THE INVENTION

The purpose of the present invention is to provide a sustained release formulation wherein a protein drug(s) as an active ingredient is encapsulated with biodegradable hydrophobic matrices.

A further purpose of the invention is to provide a sustained release formulation wherein protein drugs admixed with sulfated polysaccharides are encapsulated in biodegradable hydrophobic matrices as pharmaceutically active forms while keeping the in vivo release rate of the drug in a controlled and sustained manner for several days and weeks without high initial release.

### BRIEF DESCRIPTION OF THE DRAWINGS

FIG. 1 shows the results of complex formation between bovine serum albumin and dextran sulfate (Mw: 2,500) in various ratios at pH 4.0.

FIG. 2 shows the results of complex formation between bovine serum albumin and dextran sulfate (Mw: 4,000) in various ratios at pH 4.0.

FIG. 3 shows the results of complex formation between bovine serum albumin and dextran sulfate (Mw: 25,000) in various ratios at pH 4.0.

FIG. 4 shows the results of complex formation between bovine serum albumin and chondroitin sulfate in various ratios at pH 4.0.

FIG. 5 shows the results of complex formation between alpha-lactalbumin and dextran sulfate (Mw: 4,000) in various ratios at pH 4.0.

FIG. 6 shows the results of complex formation between ovalbumin and dextran sulfate (Mw: 4,000) in various ratios at pH 4.0.

FIG. 7 shows the results of complex formation between human growth hormone and dextran sulfate (Mw: 2,500) in various ratios at pH 4.0.

FIG. 8 shows the results of complex formation between human growth hormone and dextran sulfate (Mw: 4,000) in various ratios at pH 4.0.

FIG. 9 shows the results of complex formation between human growth hormone and chondroitin sulfate in various ratios at pH 4.0.

FIG. 10 shows the effect of pH on the complex formation between human growth hormone and dextran sulfate (Mw: 4,000).

FIG. 11 shows the effect of pH on the complex formation between human growth hormone and chondroitin sulfate.

FIGS. 12A, 12B, and 12C show the complete reversibility of complex formation between human growth hormone and dextran sulfate (Mw: 4,000) in different pHs by size exclusion chromatography. FIG. 12A shows a chromatogram of control human growth hormone; 12B shows a supernatant chromatogram of an incubation mixture of human growth hormone and dextran sulfate (Mw: 4,000) in 10 mM ammonium acetate buffer, pH 3.0, for 30 minutes; and 12C shows a chromatogram of recovered human growth hormone from a precipitated complex by adjusting pH to 7.0 with 10 mM sodium hydroxide.

FIG. 13 shows the stabilizing effect of dextran sulfate on human growth hormone by complex formation at a lower pH than the isoelectric point of the protein.

FIG. 14 shows in vitro release of proteins from microspheres containing a complex of proteins and sulfated polysaccharides.

FIG. 15 shows in vitro release of proteins from microspheres containing proteins without sulfated polysaccharides.

FIG. 16A shows a size exclusion chromatogram of standard human growth hormone. FIG. 16B shows a size exclusion chromatogram of human growth hormone extracted from microspheres prepared by the procedure described in Example 7. FIG. 16C shows a size exclusion chromatogram of human growth hormone extracted from residual microspheres after in vitro release for 5 days.

### DETAILED DESCRIPTION

As an embodiment, the present invention provides a sustained release formulation comprising protein drug, sulfated polysaccharide, and hydrophobic material, wherein the mixture of protein drug and sulfated polysaccharides is encapsulated in biodegradable hydrophobic matrices.

As another embodiment, the present invention provides a method for preparation of a sustained release protein formulation comprising a step to prepare a mixture of protein and sulfated polysaccharide;
a second step to suspend the mixture of protein and sulfated polysaccharide obtained above into a solution containing hydrophobic materials; and
a final step to remove the solvent from the suspension to get a final formulation as a solid form.

As used herein, the term “protein drug or protein” refers to peptide, protein, or a pharmaceutical composition wherein peptide or protein is incorporated as an active ingredient. Useful examples of “protein drug or protein” in the present invention include but are not limited to protein, polypeptide, and derivatives or mutants thereof obtained from natural sources, by recombinant technologies or chemical syntheses, or by modification processes such as the addition, substitution, or deletion of an amino acid or domain, or glycosilation.

Human growth hormone, growth hormone releasing hormone, growth hormone releasing peptide, interferons, colony-stimulating factors (CSFs), interleukins, macrophage activating factors, macrophage peptides, B cell factors, T cell factors, protein A, a suppressive factor of allergy, cytotoxic glycoproteins, immunotoxins, lymphotoxins, necrosis factors, tumor inhibitory factors, transforming growth factors, alpha-i antitrypsin, albumin and its fragments, apolipoprotein-E, erythropoietin, factor VII, factor VIII, factor IX, plasminogen activator, urokinase, streptokinase, protein C, C-reactive protein, renin inhibitors, collagenase inhibitors, superoxide dismutase, a platelet-derived growth factor, an epidermal growth factor, an osteogenic growth factor, bone morphogenetic protein, calcitonins, insulin, atriopeptin, a cartilage-inducing factor, connective tissue activator protein, follicle-stimulating hormone, leuteinizing hormone, leuteinizing hormone releasing hormone, nerve growth factors, parathyroid hormone, relaxin, secretin, somatomedin, an insulin-like growth factor, adrenocorticotrophic hormone, glucagon, cholecystokinin, pancreatic polypeptide, gastrin releasing peptide, a corticotropic releasing factor, thyroid stimulating hormone, monoclonal and polyclonal antibodies against various viruses, bacteria, toxins, and vaccine antigens derived from various viruses are of particular interest. More particular interests are human serum albumin, human growth hormone, interferon-alpha, erythropoietin, and a colony stimulating factor.

As used herein, the term “sulfated polysaccharide” includes the neutral form and the salt form. Particular examples are dextran sulfate, chondroitin sulfate, dermatan sulfate, heparin, heparan sulfate, and keratan sulfate.

As used herein, the term “hydrophobic materials” includes but is not limited to lipids such as fatty acids (for example, myristic acid, palmitic acid, and stearic acid); pamoic acid; monoacylglycerols (for example, glyceryl monomyristrate, glyceryl monopalmitate, and glyceryl monostearate); sorbitan fatty acid esters (for example, sorbitan myristrate, sorbitan palmitate, and sorbitan stearate); diacylglycerols; triacylglycerols (for example, trimyristin, tripalmitin, and tristearin); phospholipids (for example, phosphatidylcholine, phosphatidylethanolamine, phosphatidic acid, phosphatidylserine, phosphatidylglycerol, phosphatidylinositol, and cardiolipin); sphingosine; sphingolipids (for example, ceramides and sphinganines); waxes and their salts and derivatives. Preferable examples are stearic acid, palmitic acid, myristric acid, tristearin, glyceryl monostearate, and dipalmitoyl phosphatidic acid.

As previously mentioned, there has been a continuous demand to provide a method for encapsulating the protein drug, in its fully active state, in biodegradable hydrophobic matrices while keeping the in vivo release rate of the drug in a controlled and sustained manner for several days and weeks without high initial release. We have closely examined how proteins, particularly human growth hormone, are present in vivo so as to formulate a solution to this technical problem.

It has been known that, after being synthesized, protein hormones such as growth hormone and prolactin are stored in cellular organelles, so-called secretory granules, as highly concentrated forms and are released in soluble form by external stimuli [Dannies, P. S., Molecular and Cellular Endocrinology, 177, 87-93 (2001)]. Concentration of the synthesized proteins into granules may be extensive; prolactin, for example, is 200 times more concentrated in the dense cores of secretory granules than in the endoplasmic reticulum [Farquhar, M. G., Reid, J. J., Daniell, L. W., Endocrinology, 102, 296-311 (1978)].

We have attempted to understand how protein hormones could be present in secretory granules at such highly condensed forms, be released reversibly as soluble forms, and find a preparation method for such formulations, so-called artificial secretory granules. Therefore, it is an aim of this invention to provide a preparation method of a sustained release protein formulation mimicking artificial secretory granule.

We have systemically studied the mechanism of complex formation between proteins and sulfated polysaccharides, that is, pH effects on the complex formation, pH dependent reversibility of the complex, protein stability during the encapsulation process of the complex with hydrophobic materials such as lipids, and the release pattern of the protein from the formulation. We found that insoluble complexes were formed between proteins and sulfated polysaccharides at a lower pH than the isoelectric point of the protein, regardless of the types of proteins tested. We also observed that the protein, after being precipitated as insoluble complexes, could be recovered reversibly in soluble form only by increasing the pH to that higher than the isoelectric point of the protein. From the microparticles obtained by coating protein-sulfated polysaccharide complexes with hydrophobic materials such as lipids, protein was released continuously at a constant rate. Surprisingly, protein was released at a constant rate from the microparticles obtained by coating a mere physical mixture of protein and sulfated polysaccharides at a pH even higher than the isoelectric point of the protein.

As used herein, the term “mixture of proteins and sulfated polysaccharides” refers to a simple mixing state of proteins and sulfated polysaccharides physically, without any interaction between them or a non-covalently bonded state between them by non-covalent interaction, for example, ionic interaction, hydrophobic interaction, and hydrogen bonding. “Mixture of proteins and sulfated polysaccharides” as described above might be a liquid state in which proteins and sulfated polysaccharides were dissolved clearly or were suspended as insoluble particulates. Or, it might be a solid state as microparticles in which the solvent was removed by a drying process. Any drying method such as spray drying, freeze drying, spray freeze drying, and drying using supercritical fluid can be used. The pH of the mixture of proteins and sulfated polysaccharides is preferably lower than the isoelectric point of the protein. Insoluble complexes between proteins and sulfated polysaccharides are usually formed at a pH lower than the pI of the protein. As pH of the mixture becomes higher than pI of the protein, proteins are dissociated and released in soluble form without denaturation.

The sulfated polysaccharide is present in an amount of from about 0.01 to about 95% weight of the formulation. The preferable range is from 2.0 to 85%.

The sustained release protein formulation of this invention can further comprise protein stabilizers. Protein stabilizers can be included in the preparation step of the mixture of proteins and sulfated polysaccharides, in the suspension step of the mixture in a solution containing hydrophobic materials, or in both steps. Suitable examples of protein stabilizers are sugars, polyols, dextran, polyethyleneglycol, cyclodextrin, polyvinylalcohol, hydroxymethylcellulose, hydroxyethylcellulose, polyethyleneimine, polyvinylpyrrolidone, gelatin, collagen, albumin, surfactants, amino acids, inorganic salts, and mixtures thereof. Examples of sugars are sucrose, trehalose, maltose, mannitol, lactose, and mannose. Preferable stabilizers are glycine, trehalose, zinc chloride, mannitol, alanine, hydroxybetacyclodextrin, and polyethyleneglycol.

The sustained release formulation of protein in this invention can be preferably manufactured by preparing a mixture of proteins and sulfated polysaccharides in a liquid state or in a solid state obtained thereof by drying the liquid, suspending the mixture in the solution containing hydrophobic materials homogeneously, and removing the solvent thereof.

Solvent removal can be achieved through various methods such as spray drying, freeze drying, spray freeze drying, and drying using supercritical fluid.

The following Examples, Test Examples, and Comparative Examples are intended to further illustrate the present invention without limiting the scope of its claims in any way.

In the following Test Examples, insoluble complexes were formed between proteins and sulfated polysaccharides at a lower pH than the isoelectric point of the protein. Protein concentration was determined by size exclusion chromatography (SEC). The column was Asahipak GS-320HQ (Shodex 7.6×300 mm) and mobile phase was 10 mM phosphate buffer, pH 7.0, 150 mM NaCl. Flow rate was 0.5 ml/min and detection was UV at 215 nm.

### TEST EXAMPLE 1

### Complex Formation between Bovine Serum Albumin and Dextran Sulfate (Mw: 2,500)

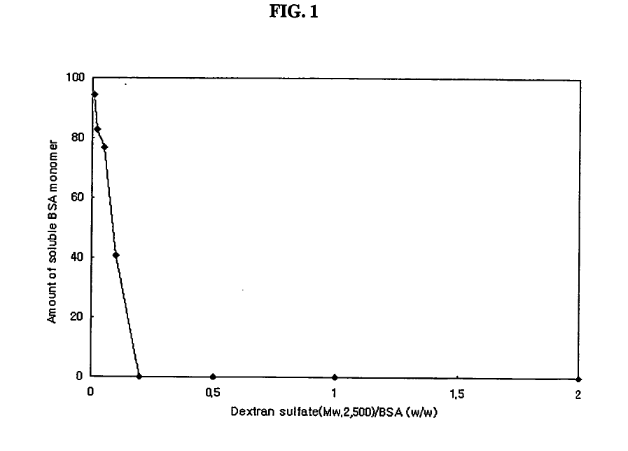

Bovine serum albumin and dextran sulfate (Mw: 2,500) were mixed in 10 mM ammonium acetate, pH 4.0, at ratios of 1:0.01, 1:0.02, 1:0.05, 1:0.1, 1:0.2, 1:0.5, 1:1, 1:2 (w/w). Protein concentration was 0.1 mg/ml. After 30 min. of incubation, the mixture was centrifuged and supernatant was removed for SEC analysis. FIG. 1 shows the results.

### TEST EXAMPLE 2

### Complex Formation between Bovine Serum Albumin and Dextran Sulfate (Mw: 4,000)

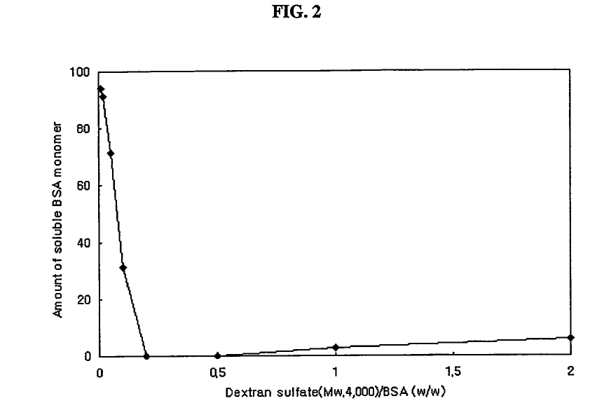

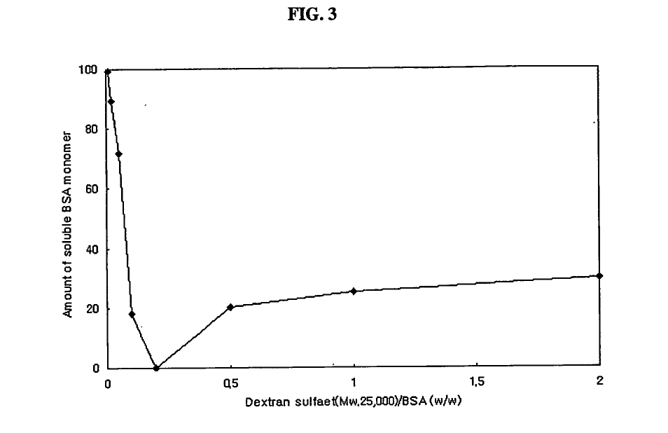

Bovine serum albumin and dextran sulfate (Mw: 4,000) were mixed in 10 mM ammonium acetate, pH 4.0, at ratios of 1:0.01, 1:0.02, 1:0.05, 1:0.1, 1:0.2, 1:0.5, 1:1, 1:2 (w/w). Protein concentration was 0.1 mg/ml. After 30 min. of incubation, the mixture was centrifuged and supernatant was removed for SEC analysis. FIG. 2 shows the results.

### TEST EXAMPLE 3

### Complex Formation between Bovine Serum Albumin and Dextran Sulfate (Mw: 25,000)

Bovine serum albumin and dextran sulfate (Mw: 4,000) were mixed in 10 mM ammonium acetate, pH 4.0, at ratios of 1:0.01, 1:0.02, 1:0.05, 1:0.1, 1:0.2, 1:0.5, 1:1, 1:2 (w/w). Protein concentration was 0.1 mg/ml. After 30 min. of incubation, the mixture was centrifuged and supernatant was removed for SEC analysis. FIG. 3 shows the results.

### TEST EXAMPLE 4

### Complex Formation between Bovine Serum Albumin and Chondroitin Sulfate

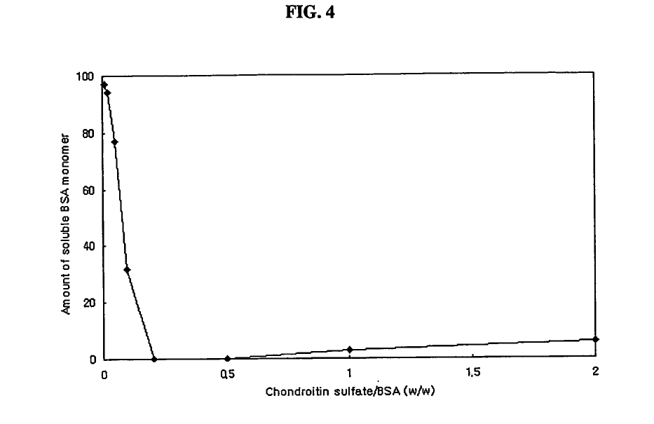

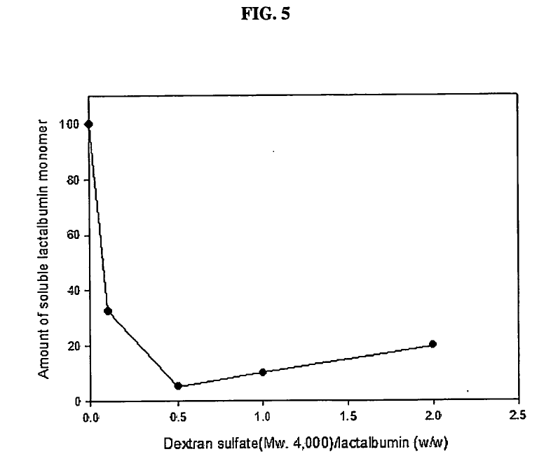

Bovine serum albumin and chondroitin sulfate were mixed in 10 mM ammonium acetate, pH 4.0, at ratios of 1:0.01, 1:0.02, 1:0.05, 1:0.1, 1:0.2, 1:0.5, 1:1, 1:2 (w/w). Protein concentration was 0.1 mg/ml. After 30 min. of incubation, the mixture was centrifuged and supernatant was removed for SEC analysis. FIG. 4 shows the results.

### TEST EXAMPLE 5

### Complex Formation between Alpha-Lactalbumin and Dextran Sulfate (Mw: 4,000)

Alpha-lactalbumin and dextran sulfate (Mw: 4,000) were mixed in 10 mM ammonium acetate, pH 4.0, at ratios of 1:0.1, 1:0.5, 1:1, 1:2 (w/w). Protein concentration was 1.0 mg/ml. After 30 min. of incubation, the mixture was centrifuged and supernatant was removed for SEC analysis. FIG. 5 shows the results.

### TEST EXAMPLE 6

### Complex Formation between Ovalbumin and Dextran Sulfate (Mw: 4,000)

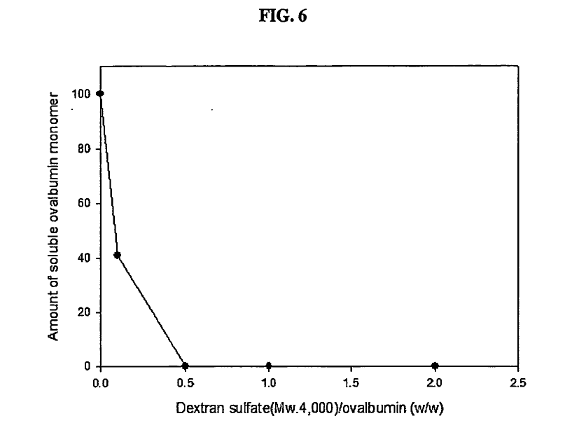

Ovalbumin and dextran sulfate (Mw: 4,000) were mixed in 10 mM ammonium acetate, pH 4.0, at ratios of 1:0.1, 1:0.5, 1:1, 1:2 (w/w). Protein concentration was 1.0 mg/ml. After 30 min. of incubation, the mixture was centrifuged and supernatant was removed for SEC analysis. FIG. 6 shows the results.

### TEST EXAMPLE 7

### Complex Formation between Human Growth Hormone and Dextran Sulfate (Mw: 2,500)

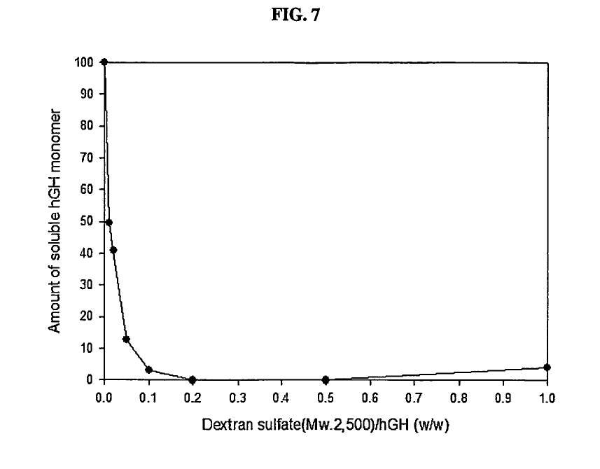

Human growth hormone and dextran sulfate (Mw: 2,500) were mixed in 10 mM ammonium acetate, pH 4.0, at ratios of 1:0.01, 1:0.02, 1:0.05, 1:0.1, 1:0.2, 1:0.5, 1:1, (w/w). Protein concentration was 0.1 mg/ml. After 30 min. of incubation, the mixture was centrifuged and supernatant was removed for SEC analysis. FIG. 7 shows the results.

### TEST EXAMPLE 8

### Complex Formation between Human Growth Hormone and Dextran Sulfate (Mw: 4,000)

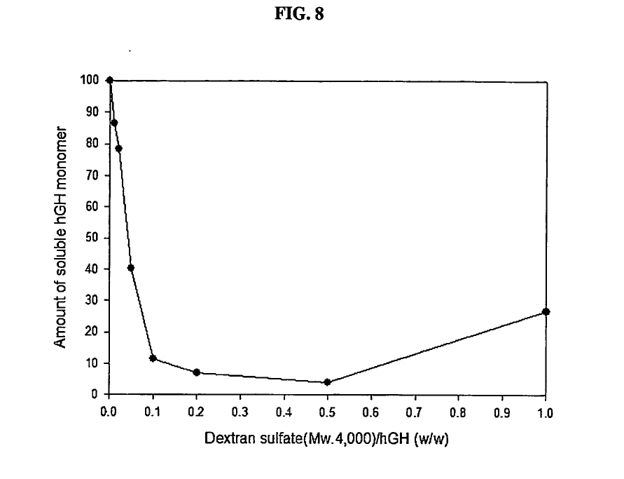

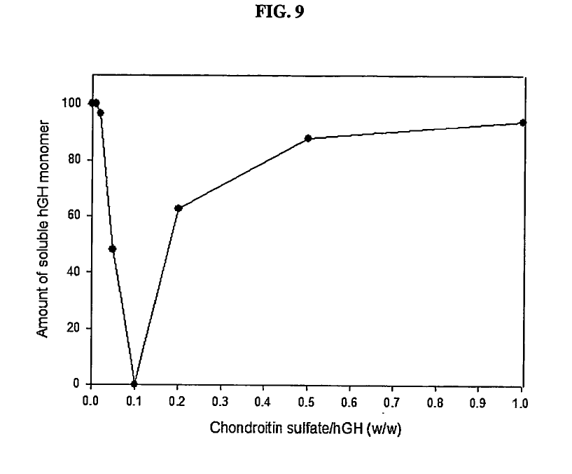

Human growth hormone and dextran sulfate (Mw: 4,000) were mixed in 10 mM ammonium acetate, pH 4.0, at ratios of 1:0.1, 1:0.2, 1:0.5, 1:1 (w/w). Protein concentration was 0.1 mg/ml. After 30 min. of incubation, the mixture was centrifuged and supernatant was removed for SEC analysis. FIG. 8 shows the results.

### TEST EXAMPLE 9

### Complex Formation between Human Growth Hormone and Chondroitin Sulfate

Human growth hormone and chondroitin sulfate were mixed in 10 mM ammonium acetate, pH 4.0, at ratios of 1:0.01, 1:0.02, 1:0.05, 1:0.1, 1:0.2, 1:0.5, 1:1 (w/w). Protein concentration was 0.1 mg/ml. After 30 min. of incubation, the mixture was centrifuged and supernatant was removed for SEC analysis. FIG. 9 shows the results.

As shown in FIGS. 1˜9, the amount of soluble protein monomer in the supernatant is inversely proportional to the amount of insoluble complexes formed between proteins and sulfated polysaccharides. Complexes were typically formed beyond a threshold ratio of sulfated polysaccharide/protein (w/w), and there was an optimal ratio of sulfated polysaccharide/protein in some cases (FIG. 9).

### TEST EXAMPLE 10

### pH Dependency of Complex Formation between Human Growth Hormone and Dextran Sulfate (Mw: 4,000)

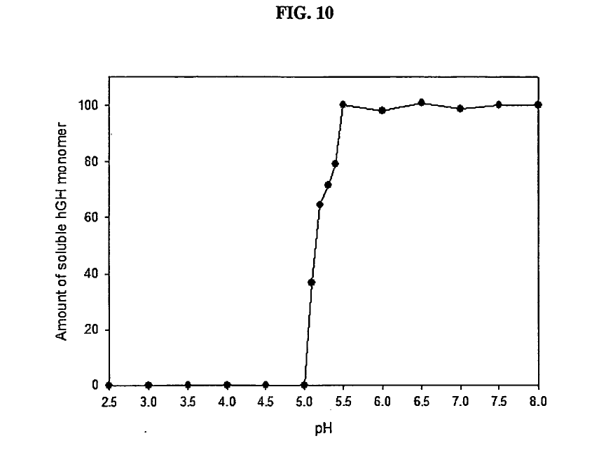

Human growth hormone and dextran sulfate (Mw: 4,000) were mixed in 10 mM ammonium acetate, pH 2.5˜8.0, at a 1:0.5 (w/w) ratio. Protein concentration was 0.1 mg/ml. After 30 min. of incubation, the mixture was centrifuged and supernatant was removed for SEC analysis. FIG. 10 shows the results.

### TEST EXAMPLE 11

### pH Dependency of Complex Formation between Human Growth Hormone and Chondroitin Sulfate

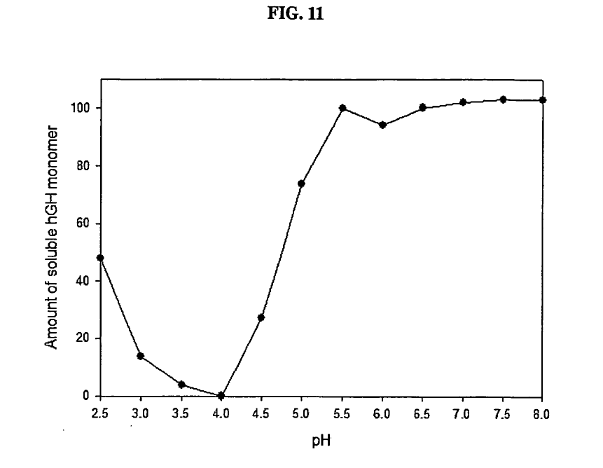

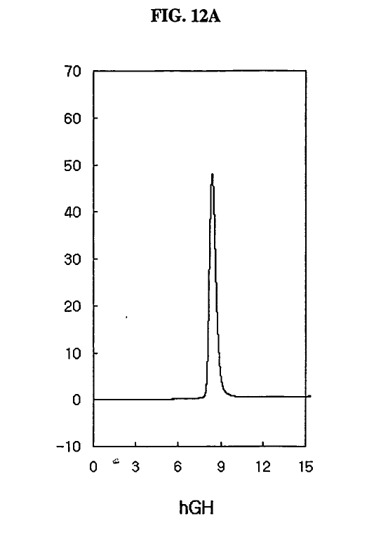

Human growth hormone and chondroitin sulfate were mixed in 10 mM ammonium acetate, pH 2.5˜8.0, at a 1:0.1 (w/w) ratio. Protein concentration was 0.1 mg/ml. After 30 min. of incubation, the mixture was centrifuged and supernatant was removed for SEC analysis. FIG. 11 shows the results.

Test Examples 1˜11 confirm that insoluble complexes were formed between protein and sulfated polysaccharide at a lower pH than the isoelectric point of the protein.

Test Example 12 confirms that the protein, after being precipitated as insoluble complexes with sulfated polysaccharides at a lower pH than isoelectric point of the protein, can be recovered reversibly in soluble form only through a change in pH. Test Example 13 also shows that the protein in the insoluble complexes with sulfated polysaccharides are stable in a harsh condition. Such a fact indicates that sulfated polysaccharides can be used as protein stabilizers by forming complexes with proteins at a lower pH than the isoelectric point of the protein.

### TEST EXAMPLE 12

### pH Dependent Reversibility of Complex Formation between Human Growth Hormone and Dextran Sulfate

Human growth hormone and dextran sulfate (Mw: 4,000) were mixed in 10 mM ammonium acetate, pH 3.0 at a 1:0.5 (w/w) ratio. Protein concentration was 0.1 mg/ml. After 30 min. of incubation, the insoluble complexes were separated from supernatant by centrifugation. The pH of the precipitate was adjusted to 7.0 with 10 mM NaOH.

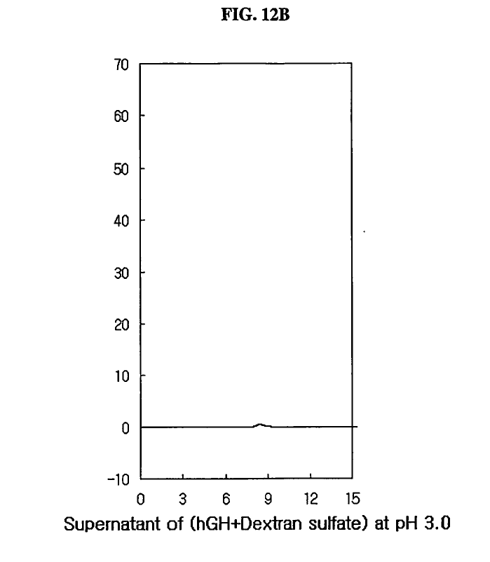

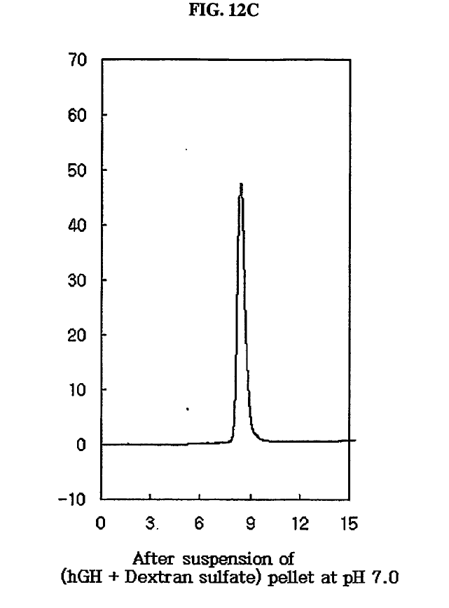

FIG. 12A shows a SEC chromatogram of standard human growth hormone of 0.1 mg/ml. FIG. 12B shows a chromatogram of supernatant after centrifugation of the incubation mixture, and FIG. 12C shows a chromatogram of recovered human growth hormone from pH-adjusted precipitate. As shown in FIGS. 12A and 12C, the amounts of standard and recovered human growth hormone were similar, and denaturation forms of protein were not detected. These results indicate that complex formation between human growth hormone and dextran sulfate occurs at a lower pH than pI of the protein, that the processes of formation/dissociation of complex are completely reversible, and that protein denaturation does not occur during the processes.

### TEST EXAMPLE 13

### Stabilization of Protein Against High Shear Stress when Forming Complex with Sulfated Polysaccharide

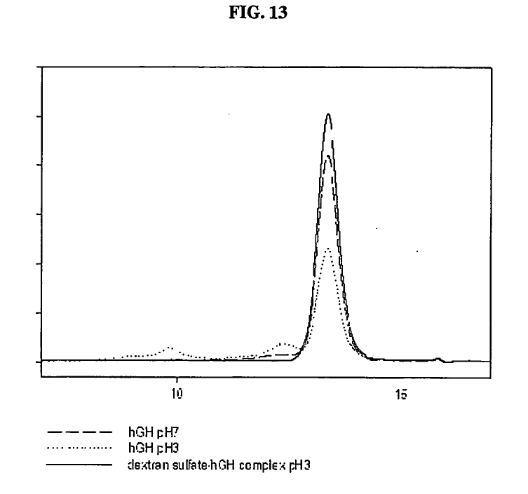

Three test solutions were prepared—a mixture of human growth hormone and dextran sulfate at pH 3.0 and human growth hormone solutions at pH 7.0 and pH 3.0. Protein concentration was 0.1 mg/ml. The test solutions were sonicated in a water bath type sonicator for 30 seconds at 4° C. After adjusting the pH of the solutions to 7.0, supernatants were separated by centrifugation for SEC analyses. FIG. 13 shows the results. As shown in the chromatograms, human growth hormone did not denature when forming complex with sulfated polysaccharide, but it denatured significantly without sulfated polysaccharides, regardless of pH. These results confirm that sulfated polysaccharides stabilize proteins against a harsh condition by forming complexes at a lower pH of the pI of the protein.

The following Examples, Comparative Examples, and Test Examples are intended to further illustrate the present invention in detail.

### EXAMPLE 1

### Preparation of Complex Particles of Bovine Serum Albumin and Dextran Sulfate (Mw: 500,000)

Bovine serum albumin and dextran sulfate (Mw: 500,000) were mixed in 0.1% (v/v) aqueous acetic acid solution. Final concentrations of protein and dextran sulfate were 3 mg/ml and 15 mg/ml. Complex particles were obtained by spray drying the above mixed solution using a Büchi-191 spray dryer at a feeding rate of 3 ml/min. Inlet temperature of the air was 85° C., and the mean diameter of particles obtained was 3.5 μm.

### EXAMPLE 2

### Preparation of Microparticles Containing Bovine Serum Albumin

Bovine serum albumin-dextran sulfate complex particles prepared by the method of Example 1 were suspended in an ethanol solution containing 5 mg/ml of stearic acid. The final solid content of the protein was 8.3% (w/w). The resulting solution was supplied to a Büchi-191 spray dryer at a feeding rate of 3 ml/min to prepare microparticles containing bovine serum albumin.

### EXAMPLE 3

### Preparation of Microparticles Containing Bovine Serum Albumin

Bovine serum albumin-dextran sulfate complex particles prepared by the method of Example 1 were suspended in an ethanol solution containing 5 mg/ml of palmitic acid. The final solid content of the protein was 8.3% (w/w). The resulting solution was supplied to a Büchi-191 spray dryer at a feeding rate of 3 ml/min to prepare microparticles containing bovine serum albumin.

### EXAMPLE 4

### Preparation of Microparticles Containing Bovine Serum Albumin

Bovine serum albumin-dextran sulfate complex particles prepared by the method of Example 1 were suspended in an ethanol solution containing 5 mg/ml of palmitic acid. The final solid content of the protein was 10% (w/w). The resulting solution was supplied to a Büchi-191 spray dryer at a feeding rate of 3 ml/min to prepare microparticles containing bovine serum albumin.

### EXAMPLE 5

### Preparation of Microparticles Containing Human Serum Albumin

Human serum albumin and dextran sulfate were mixed in 10 mM ammonium bicarbonate buffer, pH 7.0. Final concentrations of protein and dextran sulfate were 3 mg/ml and 15 mg/ml. Complex particles were obtained by spray drying the above mixed solution using a Büchi-191 spray dryer at a feeding rate of 3 ml/min. Said complex particles were suspended in an ethanol solution containing 5 mg/ml of palmitic acid. The final solid content of the protein was 8.3% (w/w). The resulting solution was supplied to a Büchi-191 spray dryer at a feeding rate of 3 ml/min to prepare microparticles containing human serum albumin.

### COMPARATIVE EXAMPLE 1

### Preparation of Bovine Serum Albumin Particles

Bovine serum albumin was dissolved in 10 mM ammonium acetate buffer, pH 4.0, at a concentration of 5 mg/ml. This solution was supplied to a Büchi-191 spray dryer at a feeding rate of 2.5 ml/min. Inlet temperature of the air was 85° C. and the mean diameter of particles obtained was 4.0 μm.

### COMPARATIVE EXAMPLE 2

### Preparation of Microparticles Containing Bovine Serum Albumin

Bovine serum albumin particles prepared by the method of

Comparative Example 1 were suspended in a methylene chloride solution containing 5 mg/ml of poly(lactic-co-glycolic acid) (RG502H from Boehringer Ingelheim). The final solid content of the protein was 10% (w/w). The resulting solution was supplied to a Büchi-191 spray dryer at a feeding rate of 3 ml/min to prepare microparticles containing bovine serum albumin.

### COMPARATIVE EXAMPLE 3

### Preparation of Microparticles Containing Bovine Serum Albumin

Bovine serum albumin particles prepared by the method of Comparative Example 1 were suspended in an ethanol solution containing 5 mg/ml of caprylate. The final solid content of the protein was 50% (w/w). The resulting solution was supplied to a Büchi-191 spray dryer at a feeding rate of 3 ml/min to prepare microparticles containing bovine serum albumin.

### COMPARATIVE EXAMPLE 4

### Preparation of Microparticles Containing Bovine Serum Albumin

Bovine serum albumin particles prepared by the method of Comparative Example 1 were suspended in an ethanol solution containing 5 mg/ml of caprylate. The final solid content of the protein was 10% (w/w). The resulting solution was supplied to a Büchi-191 spray dryer at a feeding rate of 3 ml/min to prepare microparticles containing bovine serum albumin.

### COMPARATIVE EXAMPLE 5

### Preparation of Microparticles Containing Bovine Serum Albumin

Bovine serum albumin particles prepared by the method of Comparative Example 1 were suspended in an ethanol solution containing 5 mg/ml of palmitic acid. The final solid content of the protein was 3.3% (w/w). The resulting solution was supplied to a Büchi-191 spray dryer at a feeding rate of 3 ml/min to prepare microparticles containing bovine serum albumin.

### COMPARATIVE EXAMPLE 6

### Preparation of Microparticles Containing Bovine Serum Albumin

Bovine serum albumin particles prepared by the method of Comparative Example 1 were suspended in an ethanol solution containing 5 mg/ml of palmitic acid. The final solid content of the protein was 8.3% (w/w). The resulting solution was supplied to a Büchi-191 spray dryer at a feeding rate of 3 ml/min to prepare microparticles containing bovine serum albumin.

### TEST EXAMPLE 14

### In vitro Release of Protein from Microparticles

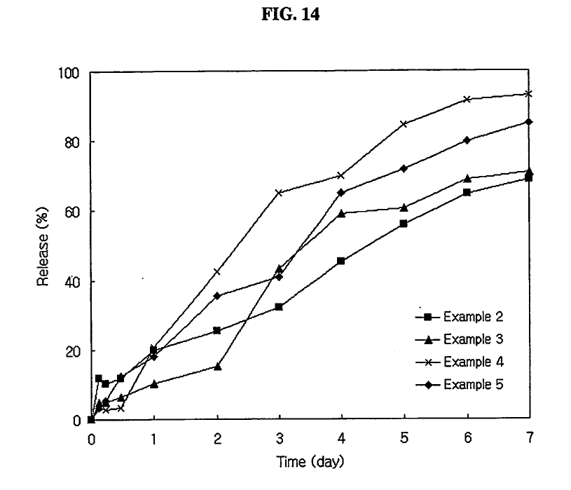

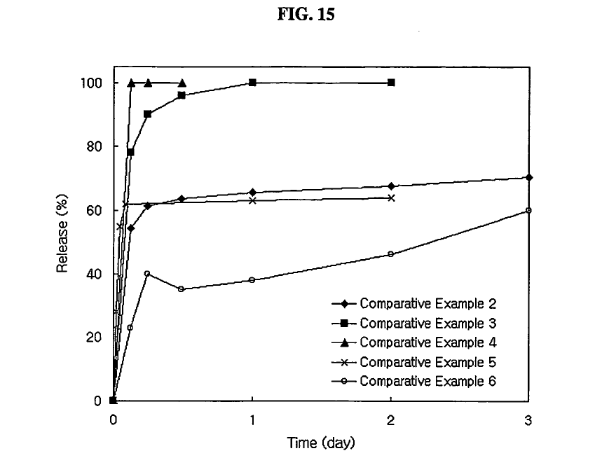

Two mg of microparticles containing proteins prepared by the methods of Examples 2˜5, Comparative Examples 2˜6 were exactly weighed and suspended in 10 mM phosphate buffer, pH 7.4. The tubes were then placed in an incubator at 37° C. At a predetermined time, the tubes were removed and centrifuged. The release protein amount in the supernatant was determined by SEC analysis, and the results were shown in FIGS. 14 and 15.

As shown in FIG. 14, from the microparticles containing proteins complexed with sulfated polysaccharides, proteins were continuously released for 7 days with a constant release rate and less than 10% of initial release. On the other hand, as shown in FIG. 15, proteins were released with a high initial release or were not released after the initial release from the microparticles containing proteins without sulfated polysaccharides.

### EXAMPLE 6

### Preparation of Complex Particles of Human Growth Hormone and Dextran Sulfate (Mw: 500,000)

Human growth hormone and dextran sulfate (Mw: 500,000) were mixed in 1.0% (v/v) aqueous acetic acid solution. Final concentrations of protein and dextran sulfate were 3 mg/ml and 15 mg/ml. Complex particles were obtained by spray drying the above mixed solution using a Büchi-191 spray dryer at a feeding rate of 3 ml/min. Inlet temperature of the air was 85° C., and the mean diameter of particles obtained was 3.2 μm.

### EXAMPLE 7

### Preparation of Microparticles Containing Human Growth Hormone

Five hundred mg of human growth hormone-dextran sulfate complex particles prepared by the method of Example 6 were suspended in 50 ml of a methylene chloride solution containing 5 mg/ml of tristearin. The resulting solution was supplied to a Büchi-191 spray dryer at a feeding rate of 3 ml/min to prepare trisearin-coated microparticles containing human growth hormone with the mean diameter of 6.5 μ.

Test Example 15 stabilities of proteins extracted from fresh microparticles and residual microparticles during in vitro release test

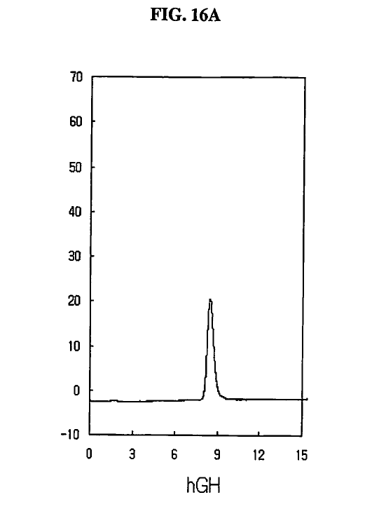

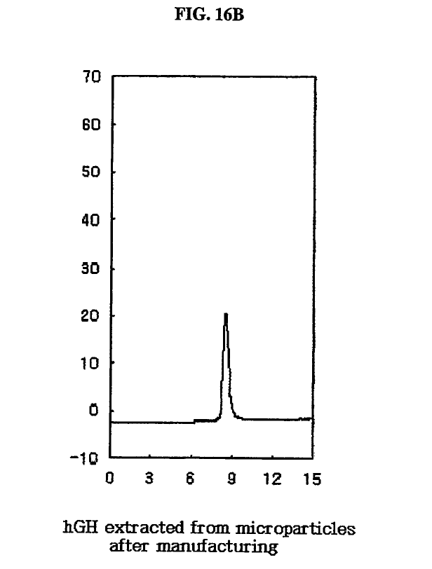

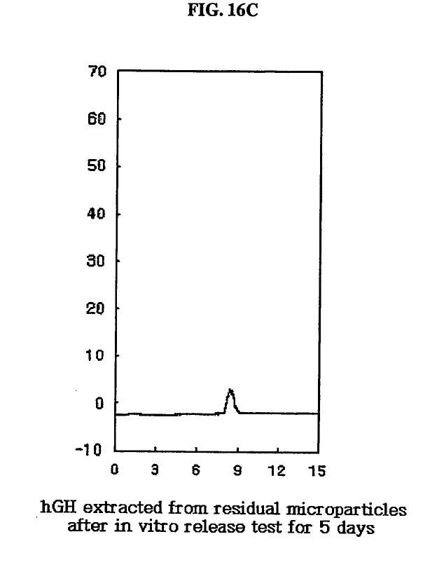

FIGS. 16B and 16C show SEC chromatograms of human growth hormone extracted from microparticles prepared by the method of Examples 7 before the releasing test and residual microparticles after the in vitro release test for 5 days.

As shown in FIGS. 16B and 16C, human growth hormone was not denatured during the microparticle manufacturing process and in vitro releasing process.

The following Examples are intended to further illustrate preparation methods of a protein-containing sustained release formulation according to the present invention. However, the scope of its claims will not be limited to the examples described previously or accordingly.

### EXAMPLE 8

### Preparation of Complex Particles of Bovine Serum Albumin and Dextran Sulfate (Mw: 2,500)

Bovine serum albumin and dextran sulfate (Mw: 2,500) were mixed in 10 mM ammonium acetate buffer, pH 4.0. Final concentrations of protein and dextran sulfate were 5 mg/ml and 1 mg/ml. Complex particles were obtained by spray drying the above mixed solution using a Büchi-191 spray dryer at a feeding rate of 5 ml/min. Inlet temperature of the air was 85° C., and the mean diameter of particles obtained was 4.0 μm.

### EXAMPLE 9

### Preparation of Complex Particles of Bovine Serum Albumin and Heparan Sulfate

Bovine serum albumin and heparan sulfate were mixed in 50 mM phosphate buffer, pH 3.0. Final concentrations of protein and heparan sulfate were 5 mg/ml and 1 mg/ml. Complex particles were obtained by spray drying the above mixed solution using a Büchi-191 spray dryer at a feeding rate of 5 ml/min. Inlet temperature of the air was 85° C., and the mean diameter of particles obtained was 4.0 μm.

### EXAMPLE 10

### Preparation of Complex Particles of Bovine Serum Albumin and Dextran Sulfate (Mw: 500,000)

Bovine serum albumin and dextran sulfate (Mw: 500,000) were mixed in 50% aqueous acetic acid solution. Final concentrations of protein and dextran sulfate were 5 mg/ml and 1 mg/ml. Complex particles were obtained by spray drying the above mixed solution using a Büchi-191 spray dryer at a feeding rate of 5 ml/min. Inlet temperature of the air was 60° C., and the mean diameter of particles obtained was 4.5 μm.

### EXAMPLE 11

### Preparation of Complex Particles of Bovine Serum Albumin and Heparin

Bovine serum albumin and heparin were mixed in 5 mM phosphate buffer, pH 4.0. Final concentrations of protein and heparin were 3 mg/ml and 15 mg/ml. Complex particles were obtained by spray drying the above mixed solution using a Büchi-191 spray dryer at a feeding rate of 3 ml/min. Inlet temperature of the air was 85° C., and the mean diameter of particles obtained was 3.5 μm.

### EXAMPLE 12

### Preparation of Complex Particles of Human Serum Albumin and Dextran Sulfate (Mw: 500,000)

Human serum albumin and dextran sulfate (Mw: 500,000) were mixed in 0.1% aqueous acetic acid solution. Final concentrations of protein and dextran sulfate were 3 mg/ml and 15 mg/ml. Complex particles were obtained by spray drying the above mixed solution using a Büchi-191 spray dryer at a feeding rate of 3 ml/min. Inlet temperature of the air was 85° C., and the mean diameter of particles obtained was 3.8 μm.

### EXAMPLE 13

### Preparation of Complex Particles of Human Serum Albumin and Dextran Sulfate (Mw: 500,000) with Protein Stabilizer

Human serum albumin, dextran sulfate (Mw: 500,000), and glycine were mixed in 10 mM ammonium acetate buffer, pH 5.0, at a ratio of 1:5:4 (w/w/w) with a final protein concentration of 5 mg/ml. Complex particles containing a protein stabilizer were obtained by spray drying the above mixed solution using a Büchi-191 spray dryer at a feeding rate of 3 ml/min. Inlet temperature of the air was 105° C., and the mean diameter of particles obtained was 3.0 μm.

### EXAMPLE 14

### Preparation of Complex Particles of Human Serum Albumin and Heparin with Protein Stabilizer

Human serum albumin, heparin, and trehalose were mixed in 10 mM ammonium acetate buffer (pH 5.0) containing 0.05% (w/v) Tween 80, at a ratio of 1:5:4 (w/w/w) with a final protein concentration of 5 mg/ml. Complex particles containing protein stabilizer were obtained by spray drying the above mixed solution using a Büchi-191 spray dryer at a feeding rate of 3 ml/min. Inlet temperature of the air was 105° C., and the mean diameter of particles obtained was 3.0 μm.

### EXAMPLE 15

### Preparation of Complex Particles of Human Growth Hormone and Heparan Sulfate

Human growth hormone and heparan sulfate were mixed in 10 mM ammonium acetate buffer, pH 4.0. Final concentrations of protein and heparan sulfate were 5 mg/ml and 1 mg/ml. Complex particles were obtained by spray drying the above mixed solution using a Büchi-191 spray dryer at a feeding rate of 3 ml/min. Inlet temperature of the air was 85° C., and the mean diameter of particles obtained was 4.0 μm.

### EXAMPLE 16

### Preparation of Complex Particles of Human Growth Hormone and Dextran Sulfate (Mw: 2,500)

Human growth hormone and dextran sulfate (Mw: 2,500) were mixed in 50 mM phosphate buffer, pH 4.0. Final concentrations of protein and dextran sulfate were 5 mg/ml and 1 mg/ml. Complex particles were obtained by spray drying the above mixed solution using a Büchi-191 spray dryer at a feeding rate of 5 ml/min. Inlet temperature of the air was 85° C., and the mean diameter of particles obtained was 3.8 μm.

### EXAMPLE 17

### Preparation of Complex Particles of Human Growth Hormone and Dextran Sulfate (Mw: 500,000) with Protein Stabilizer

A ternary mixed solution of human growth hormone, dextran sulfate (Mw: 500,000), and zinc chloride was prepared in 50% aqueous acetic acid with final concentrations of 3 mg/ml, 15 mg/ml, and 0.02 mg/ml, respectively.

Complex particles were obtained by spray drying the above mixed solution using a Büchi-191 spray dryer at a feeding rate of 4 ml/min. Inlet temperature of the air was 90° C., and the mean diameter of particles obtained was 4.0 μm.

### EXAMPLE 18

### Preparation of Complex Particles of Human Growth Hormone and Chondroitin Sulfate

Human growth hormone and chondroitin sulfate were mixed in 0.1% aqueous acetic acid solution. Final concentrations of protein and chondroitin sulfate were 3 mg/ml and 15 mg/ml. Complex particles were obtained by spray drying the above mixed solution using a Büchi-191 spray dryer at a feeding rate of 3 ml/min. Inlet temperature of the air was 85° C., and the mean diameter of particles obtained was 3.0 μm.

### EXAMPLE 19

### Preparation of Complex Particles of Human Growth Hormone and Dermatan Sulfate

Human Growth hormone and dermatan sulfate were mixed in 0.1% aqueous acetic acid solution. Final concentrations of protein and dermatan sulfate were 3 mg/ml and 20 mg/ml. Complex particles were obtained by spray drying the above mixed solution using a Büchi-191 spray dryer at a feeding rate of 3 ml/min. Inlet temperature of the air was 85° C. and the mean diameter of particles obtained was 4.0 μm.

### EXAMPLE 20

### Preparation of Complex Particles of Human Growth Hormone and Keratan Sulfate

Human growth hormone and keratan sulfate were mixed in 0.1% aqueous acetic acid solution. Final concentrations of protein and keratan sulfate were 3 mg/ml and 15 mg/ml. Complex particles were obtained by spray drying the above mixed solution using a Büchi-191 spray dryer at a feeding rate of 3 ml/min. Inlet temperature of the air was 85° C., and the mean diameter of particles obtained was 3.8 μm.

### EXAMPLE 21

### Preparation of Complex Particles of Interferon-Alpha and Dextran Sulfate (Mw: 500,000)

Interferon-alpha containing complex particles were obtained by spray drying a mixed solution of interferon-alpha and dextran sulfate (Mw: 500,000) at a ratio of 1:59 (w/w) using a Büchi-191 spray dryer at a feeding rate of 3 ml/min. Inlet temperature of the air was 85° C., and the mean diameter of particles obtained was 3.0 μm.

### EXAMPLE 22

### Preparation of Complex Particles of Interferon-Alpha and Dextran Sulfate (Mw: 500,000) with Protein Stabilizer

Interferon-alpha containing complex particles were obtained by spray drying a ternary mixed solution of interferon-alpha, human serum albumin, and dextran sulfate (Mw: 500,000) at a ratio of 1:9:50 (w/w/w) using a Büchi-191 spray dryer at a feeding rate of 3 ml/min. Inlet temperature of the air was 95° C., and the mean diameter of particles obtained was 3.5 μm.

### EXAMPLE 23

### Preparation of Complex Particles of Interferon-Alpha and Dextran Sulfate (Mw: 500,000) with Protein Stabilizer

Interferon-alpha containing complex particles were obtained by spray drying a ternary mixed solution of interferon-alpha, mannitol, and dextran sulfate (Mw: 500,000) at a ratio of 1:9:50 (w/w/w) using a Büchi-191 spray dryer at a feeding rate of 3 ml/min. Inlet temperature of the air was 105° C., and the mean diameter of particles obtained was 4.5 μm.

### EXAMPLE 24

### Preparation of Complex Particles of interferon-Alpha and Keratran Sulfate with Protein Stabilizer

Interferon-alpha containing complex particles were obtained by spray drying a ternary mixed solution of interferon-alpha, glycine, and keratan sulfate at a ratio of 1:4:5 (w/w/w) using a Büchi-191 spray dryer at a feeding rate of 2.5 ml/min. Inlet temperature of the air was 105° C., and the mean diameter of particles obtained was 3 μm.

### EXAMPLE 25

### Preparation of Complex Particles of Erythropoietin and Dextran Sulfate (Mw: 500,000)

Erythropoietin and dextran sulfate (Mw: 500,000) were mixed in 0.1% aqueous acetic acid solution at a protein:polysaccharide ratio of 1:5 (w/w).

Complex particles were obtained by spray drying the above mixed solution using a Büchi-191 spray dryer at a feeding rate of 3 ml/min. Inlet temperature of the air was 85° C., and the mean diameter of particles obtained was 3.0 μm.

### EXAMPLE 26

### Preparation of Complex Particles of Granulocyte-Colony Stimulating Factor and Dextran Sulfate (Mw: 500,000)

Granulocyte-colony stimulating factor and dextran sulfate (Mw: 500,000) were mixed in 0.1% aqueous acetic acid solution at a protein:polysaccharide ratio of 1:5 (w/w). Complex particles were obtained by spray drying the above mixed solution using a Büchi-191 spray dryer at a feeding rate of 3 ml/min. Inlet temperature of the air was 85° C., and the mean diameter of particles obtained was 3.0 μm.

### EXAMPLE 27

### Preparation of Microparticles Containing Bovine Serum Albumin

Five hundred mg of bovine serum albumin-dextran sulfate complex particles prepared by the method of Example 1 were suspended in 50 ml ethanol solution containing 10 mg/ml of myristic acid. The resulting solution was supplied to a Büchi-191 spray dryer at a feeding rate of 3 ml/min to prepare lipid-coated microparticles containing bovine serum albumin. The mean diameter of particles obtained was 7.0 μm.

### EXAMPLE 28

### Preparation of Microparticles Containing Bovine Serum Albumin

Five hundred mg of bovine serum albumin-dextran sulfate complex particles prepared by the method of Example 1 were suspended in 50 ml ethanol solution containing 10 mg/ml of stearic acid. The resulting solution was supplied to a Büchi-191 spray dryer at a feeding rate of 3 ml/min to prepare lipid-coated microparticles containing bovine serum albumin. The mean diameter of particles obtained was 6.3 μm.

### EXAMPLE 29

### Preparation of Microparticles Containing Bovine Serum Albumin

Five hundred mg of bovine serum albumin-dextran sulfate complex particles prepared by the method of Example 1 were suspended in 50 ml ethanol solution containing 10 mg/ml of Span 60. The resulting solution was supplied to a Büchi-191 spray dryer at a feeding rate of 3 ml/min to prepare Span 60-coated microparticles containing bovine serum albumin. The mean diameter of particles obtained was 5.0 μm.

### EXAMPLE 30

### Preparation of Microparticles Containing Human Growth Hormone

Five hundred mg of human growth hormone-chondroitin sulfate complex particles prepared by the method of Example 18 were suspended in 50 ml ethanol solution containing 5 mg/ml of palmitic acid. The resulting solution was supplied to a Büchi-191 spray dryer at a feeding rate of 3 ml/min to prepare lipid-coated microparticles. The mean diameter of particles obtained was 6.3 μm.

### EXAMPLE 31

### Preparation of Microparticles Containing Human Growth Hormone

Five hundred mg of human growth hormone-dextran sulfate complex particles prepared by the method of Example 6 were suspended in 50 ml ethanol solution containing 5 mg/ml of stearic acid. The resulting solution was supplied to a Büchi-191 spray dryer at a feeding rate of 3 ml/min to prepare lipid-coated microparticles. The mean diameter of particles obtained was 6.0 μm.

### EXAMPLE 32

### Preparation of Microparticles Containing Human Growth Hormone

Five hundred mg of human growth hormone-dextran sulfate complex particles prepared by the method of Example 6 were suspended in 50 ml ethanol solution containing 5 mg/ml of glyceryl monostearate. The resulting solution was supplied to a Büchi-191 spray dryer at a feeding rate of 3 ml/min to prepare lipid-coated microparticles. The mean diameter of particles obtained was 5.5 μm.

### EXAMPLE 33

### Preparation of Microparticles Containing Human Growth Hormone

Five hundred mg of human growth hormone-dermatan sulfate complex particles prepared by the method of Example 19 were suspended in 50 ml chloroform solution containing 5 mg/ml of dipalmitoyl phosphatidic acid. The resulting solution was supplied to a Büchi-191 spray dryer at a feeding rate of 3 ml/min to prepare lipid-coated microparticles. The mean diameter of particles obtained was 5.3 μm.

### EXAMPLE 34

### Preparation of Microparticles Containing Human Growth Hormone

Five hundred mg of human growth hormone-keratan sulfate complex particles prepared by the method of Example 20 were suspended in 50 ml ethanol solution containing 10 mg/ml of distearoyl phosphatidylcholine. The resulting solution was supplied to a Büchi-191 spray dryer at a feeding rate of 3 ml/min to prepare lipid-coated microparticles. The mean diameter of particles obtained was 6.2 μm.

### EXAMPLE 35

### Preparation of Microparticles Containing interferon-Alpha

Two hundred fifty mg of interferon alpha-dextran sulfate complex particles prepared by the method of Example 21 were suspended in 25 ml ethanol solution containing 10 mg/ml of palmitic acid. The resulting solution was supplied to a Büchi-191 spray dryer at a feeding rate of 3 ml/min to prepare lipid-coated microparticles. The mean diameter of particles obtained was 5.0 μm.

### EXAMPLE 36

### Preparation of Microparticles Containing Interferon-Alpha

Two hundred fifty mg of interferon alpha-dextran sulfate complex particles prepared by the method of Example 23 were suspended in 250 ml ethanol solution containing 10 mg/ml of glyceryl monostearate. The resulting solution was supplied to a Büchi-191 spray dryer at a feeding rate of 3 ml/min to prepare lipid-coated microparticles. The mean diameter of particles obtained was 6.4 μm.

### EXAMPLE 37

### Preparation of Microparticles Containing Erythropoietin

Two hundred fifty mg of erythropoietin-dextran sulfate complex particles prepared by the method of Example 25 were suspended in 25 ml ethanol solution containing 10 mg/ml of stearic acid. The resulting solution was supplied to a Büchi-191 spray dryer at a feeding rate of 3 ml/min to prepare lipid-coated microparticles. The mean diameter of particles obtained was 5.0 μm.

### EXAMPLE 38

### Preparation of Microparticles Containing Granulocyte-Colony Stimulating Factor

Two hundred fifty mg of granulocyte-colony stimulating factor-dextran sulfate complex particles prepared by the method of Example 26 were suspended in 25 ml ethanol solution containing 10 mg/ml of palmitic acid. The resulting solution was supplied to a Büchi-191 spray dryer at a feeding rate of 3 ml/min to prepare lipid-coated microparticles. The mean diameter of particles obtained was 5.0 μm.

### EXAMPLE 39

### Preparation of Microparticles Containing Human Growth Hormone

Human growth hormone and dextran sulfate (Mw: 500,000) were mixed in 10 mM ammonium acetate buffer, pH 4.0, at a protein:polysaccharide ratio of 1:2 (w/w). The solution was mixed with an ethanol solution containing 5 mg/ml of glyceryl monostearate. The resulting mixed solution was applied to a Büchi-191 spray dryer at a feeding rate of 3 ml/min to prepare lipid-coated microparticles. Inlet temperature of the air was 85° C., and the mean diameter of particles obtained was 5.2 μm.

### EXAMPLE 40

### Preparation of Microparticles Containing Human Growth Hormone

Human growth hormone and dextran sulfate (Mw: 500,000) were mixed in 10 mM ammonium acetate buffer, pH 4.0, at a protein:polysaccharide ratio of 1:5 (w/w). The solution was mixed with an ethanol solution containing 5 mg/ml of glyceryl monostearate. The resulting mixed solution was applied to a Büchi-191 spray dryer at a feeding rate of 3 ml/min to prepare lipid-coated microparticles. Inlet temperature of the air was 85° C., and the mean diameter of particles obtained was 4.8 μm.

### EXAMPLE 41

### Preparation of Microparticles Containing Human Growth Hormone

Human growth hormone and chondroitin sulfate were mixed in 10 mM ammonium acetate buffer, pH 4.0, at a protein:polysaccharide ratio of 1:2 (w/w). This solution was mixed with an ethanol solution containing 5 mg/ml of stearic acid. The resulting mixed solution was applied to a Büchi-191 spray dryer at a feeding rate of 3 ml/min to prepare lipid-coated microparticles. Inlet temperature of the air was 85° C., and the mean diameter of particles obtained was 4.8 μm.

### EXAMPLE 42

### Preparation of Microparticles Containing Human Growth Hormone

Human growth hormone and keratan sulfate were mixed in 0.1% aqueous acetic acid solution at a protein:polysaccharide ratio of 1:2 (w/w). The solution was mixed with an ethanol solution containing 5 mg/ml of palmitic acid. The resulting mixed solution was applied to a Büchi-191 spray dryer at a feeding rate of 3 ml/min to prepare lipid-coated microparticles. Inlet temperature of the air was 80° C., and the mean diameter of particles obtained was 5.1 μm.

### EXAMPLE 43

### Preparation of Microparticles Containing Human Growth Hormone

Human growth hormone and heparan sulfate were mixed in 10 mM ammonium acetate buffer, pH 4.0, at a protein:polysaccharide ratio of 1:1 (w/w). The solution was mixed with an ethanol solution containing 5 mg/ml of stearic acid. The resulting mixed solution was applied to a Büchi-191 spray dryer at a feeding rate of 3 ml/min to prepare lipid-coated microparticles. Inlet temperature of the air was 75° C., and the mean diameter of particles obtained was 5.2 μm.

### EXAMPLE 44

### Preparation of Microparticles Containing Interferon-Alpha

Interferon-alpha and dextran sulfate were mixed in 10 mM ammonium acetate buffer, pH 5.0, at a protein:polysaccharide ratio of 1:1 (w/w). The solution was mixed with an ethanol solution containing 5 mg/ml of glyceryl monostearate. The resulting mixed solution was applied to a Büchi-191 spray dryer at a feeding rate of 3 ml/min to prepare lipid-coated microparticles. Inlet temperature of the air was 85° C., and the mean diameter of particles obtained was 4.7 μm.

### EXAMPLE 45

### Preparation of Microparticles Containing Interferon-Alpha

Interferon-alpha, chondroitin sulfate, and alanine were mixed in 10 mM ammonium acetate buffer, pH 4.0, at a ratio of 1:1:5 (w/w/w). The solution was mixed with an ethanol solution containing 5 mg/ml of glyceryl monostearate. The resulting mixed solution was applied to a Büchi-191 spray dryer at a feeding rate of 3 ml/min to prepare lipid-coated microparticles. Inlet temperature of the air was 85° C., and the mean diameter of particles obtained was 5.0 μm.

### EXAMPLE 46

### Preparation of Microparticles Containing Interferon-Alpha

Interferon-alpha, dermatan sulfate, and hydroxypropyl-beta-cyclodextrin were mixed in 10 mM ammonium acetate buffer, pH 5.0, at a ratio of 1:1:2 (w/w/w). The solution was mixed with ethanol solution containing 5 mg/ml of stearic acid. The resulting mixed solution was applied to a Büchi-191 spray dryer at a feeding rate of 3 ml/min to prepare lipid-coated microparticles. Inlet temperature of the air was 85° C., and the mean diameter of particles obtained was 4.5 μm.

### EXAMPLE 47

### Preparation of Microparticles Containing Interferon-Alpha

Interferon-alpha, keratan sulfate, and trehalose were mixed in 10 mM ammonium acetate buffer, pH 5.0, at a ratio of 1:2:5 (w/w/w). The solution was mixed with an ethanol solution containing 5 mg/ml of palmitic acid. The resulting mixed solution was applied to a Büchi-191 spray dryer at a feeding rate of 3 ml/min to prepare lipid-coated microparticles. Inlet temperature of the air was 85° C., and the mean diameter of particles obtained was 5.8 μm.

### EXAMPLE 48

### Preparation of Microparticles Containing Bovine Serum Albumin

Complex particles of bovine serum albumin-dextran sulfate were prepared by spraying 0.1% aqueous acetic acid solution containing protein and polysaccharide at a ratio of 1:1 (w/w) onto liquid nitrogen, followed by freeze drying. Five hundred mg of the particles obtained were suspended in an ethanol solution containing 5 mg/ml of palmitic acid. The resulting suspended solution was applied to a Büchi-191 spray dryer at a feeding rate of 2.5 ml/min to prepare lipid-coated microparticles. Inlet temperature of the air was 85° C., and the mean diameter of particles obtained was 4.0 μm.

### EXAMPLE 49

### Preparation of Microparticles Containing Bovine Serum Albumin

Complex particles of bovine serum albumin-chondroitin sulfate were prepared by spraying 1.0% aqueous acetic acid solution containing protein and polysaccharide at a ratio of 1:1 (w/w) onto liquid nitrogen, followed by freeze drying. Five hundred mg of the particles obtained were suspended in an ethanol solution containing 5 mg/ml of palmitic acid. The resulting suspended solution was applied to a Büchi-191 spray dryer at a feeding rate of 2.5 ml/min to prepare lipid-coated microparticles. Inlet temperature of the air was 85° C., and the mean diameter of particles obtained was 4.8 μm.

### EXAMPLE 50

### Preparation of Microparticles Containing Bovine Serum Albumin

Complex particles of bovine serum albumin-dermatan sulfate were prepared by spraying 10 mM ammonium acetate buffer (pH 5.0) containing protein and polysaccharide at a ratio of 1:1 (w/w) onto liquid nitrogen, followed by freeze drying. Five hundred mg of the particles obtained were suspended in an ethanol solution containing 5 mg/ml of palmitic acid. The resulting suspended solution was applied to a Büchi-191 spray dryer at a feeding rate of 2.5 ml/min to prepare lipid-coated microparticles. Inlet temperature of the air was 85° C., and the mean diameter of particles obtained was 5.8 μm.

### EXAMPLE 51

### Preparation of Microparticles Containing Human Growth Hormone

Complex particles of human growth hormone-dextran sulfate were prepared by spraying 10 mM ammonium acetate buffer (pH 4.0) containing protein and polysaccharide at a ratio of 1:1 (w/w) onto liquid nitrogen, followed by freeze drying. Five hundred mg of the particles obtained were suspended in an ethanol solution containing 10 mg/ml of palmitic acid. The resulting suspended solution was applied to a Bëchi-191 spray dryer at a feeding rate of 3 ml/min to prepare lipid-coated microparticles. Inlet temperature of the air was 85° C., and the mean diameter of particles obtained was 6.2 μm.

### EXAMPLE 52

### Preparation of Microparticles Containing Interferon-Alpha

Complex particles of interferon alpha-dextran sulfate-polyethyleneglycol were prepared by spraying 10 mM ammonium acetate buffer (pH 4.0) containing protein, sulfated polysaccharide, and PEG at a ratio of 1:1:2 (w/w/w) onto liquid nitrogen, followed by freeze drying. Fifty mg of the particles obtained were suspended in an ethanol solution containing 10 mg/ml of palmitic acid. The resulting suspended solution was applied to a Büchi-191 spray dryer at a feeding rate of 2.5 ml/min to prepare lipid-coated microparticles. Inlet temperature of the air was 85° C., and the mean diameter of particles obtained was 5.8 μm. EXAMPLE 53

### Preparation of Microparticles Containing Erythropoietin

Complex particles of erythropoietin-dextran sulfate-sucrose were prepared by spraying 1% aqueous acetic acid solution containing protein, polysaccharide, and disaccharide at a ratio of 1:1:5 (w/w/w) onto liquid nitrogen, followed by freeze drying. Fifty mg of the particles obtained were suspended in an ethanol solution containing 5 mg/ml of stearic acid. The resulting suspended solution was applied to a Büchi-191 spray dryer at a feeding rate of 3 ml/min to prepare lipid-coated microparticles. Inlet temperature of the air was 85° C., and the mean diameter of particles obtained was 4.9 μm.

### APPLICATION IN THE PHARMACEUTICAL INDUSTRY

As previously described, a sustained release formulation can be prepared by the method of the present invention, wherein protein drugs are encapsulated in biodegradable hydrophobic matrices as pharmaceutically active forms by forming complexes with sulfated polysaccharides. Further, a sustained release formulation can be obtained by the encapsulation of a mere mixture of protein and sulfated polysaccharide, without the formation of a complex, in hydrophobic materials. The sustained release formulation prepared by the present invention can be used to effectively treat a disease by keeping the concentration of the pharmaceutically active protein drug at a sufficiently high level for a long period when injected in vivo once.

1. A sustained release solid formulation characterized by comprising protein drug, sulfated polysaccharide, and hydrophobic material, wherein the mixture of protein and sulfated polysaccharide are encapsulated with hydrophobic material.

2. The formulation of claim 1 wherein said sulfated polysaccharide is selected from the group of dextran sulfate, chondroitin sulfate, dermatan sulfate, heparin, heparan sulfate, and keratan sulfate.

3. The formulation of claim 1 wherein said hydrophobic material is selected from the group of fatty acids, pamoic acid, monoacyl glycerols, sorbitan fatty acid esters, diacyl glycerols, triglycerides, phospholipids, sphingosines, sphingolipids, waxes, and salts or derivatives thereof.

4. The formulation of claim 1 wherein said sulfated polysaccharide is present in an amount of from 0.01 to 95% weight of the formulation.

5. The formulation of claim 1 wherein the composition further comprises protein stabilizers.

6. The formulation of claim 5 wherein said protein stabilizer is selected from the group of sucrose, trehalose, maltose, mannitol, lactose, mannose, polyol, dextran, polyethyleneglycol, cyclodextrin, polyvinylalcohol, hydroxypropylmethylcellulose, hydroxyethylcellulose, polyethyleneimine, polyvinylpyrrolidone, gelatin, collagen, albumin, surfactants, amino acids, inorganic salts, and mixtures thereof.

7. A process to prepare a sustained release solid formulation characterized by comprising a step to prepare a mixture of proteins and sulfated polysaccharides, a step to suspend the mixture obtained in the solution containing hydrophobic materials, and a step to remove the solvent from the suspension to obtain a solid formulation.

8. The process of claim 7 wherein said sulfated polysaccharide is selected from dextran sulfate, chondroitin sulfate, dermatan sulfate, heparin, heparan sulfate, and keratan sulfate.

9. The process of claim 7 wherein said hydrophobic material is selected from the group consisting of fatty acids, pamoic acid, monoacyl glycerols, sorbitan fatty acid esters, diacyl glycerols, triglycerides, phospholipids, sphingosines, sphingolipids, waxes, and salts or derivatives thereof.

10. The process of claim 7 wherein said sulfated polysaccharide is present in an amount of from 0.01 to 95% weight of the formulation.

11. The process of claim 7 wherein the mixture of protein and sulfated polysaccharide is a solid microparticulate form.

12. The process of claim 11 wherein said solid microparticulate is prepared by drying the liquid mixture of protein and sulfated polysaccharide.

13. The process of claim 12 wherein said solid microparticulate is obtained by spray drying, freeze drying, spray freeze drying, and drying using supercritical fluid.

14. The process of claim 7 wherein the mixture of protein and sulfated polysaccharide is a liquid state.

15. The process of claim 7 wherein the pH of the mixture of protein and sulfated polysaccharide is lower than the isoelectric point of the protein.

16. The process of claim 7 wherein the process further comprises a step to add protein stabilizers.

17. The process of claim 16 wherein said protein stabilizer is selected from the group of sucrose, trehalose, maltose, mannitol, lactose, mannose, polyol, dextran, polyethyleneglycol, cyclodextrin, polyvinylalcohol, hydroxypropylmethylcellulose, hydroxyethylcellulose, polyethyleneimine, polyvinylpyrrolidone, gelatin, collagen, albumin, surfactants, amino acids, inorganic salts, and mixtures thereof.

18. The process of claim 11 wherein the pH of the mixture of protein and sulfated polysaccharide is lower than the isoelectric point of the protein.

19. The process of claim 14 wherein the pH of the mixture of protein and sulfated polysaccharide is lower than the isoelectric point of the protein.
